(19) United States
(12) Patent Application Publication
(10) Pub. No.: US 2025/0359808 A1
(43) Pub. Date: Nov. 27, 2025

(54) ELECTROMYOGRAPHY DEVICES AND METHODS INCLUDING MAPPING BETWEEN SPATIAL MUSCLE ACTIVITY AND ELECTROMYOGRAPHY DATA

(71) Applicant: Battelle Memorial Institute, Columbus, OH (US)

(72) Inventors: Nicholas J. Tacca, Columbus, OH (US); Eric Meyers, Columbus, OH (US); Ian W. Baumgart, Albany, OH (US)

(21) Appl. No.: 19/215,481

(22) Filed: May 22, 2025

Related U.S. Application Data
(60) Provisional application No. 63/650,471, filed on May 22, 2024.

(51) Int. Cl. A61B 5/389; A61B 5/00; A61B 5/256; A61B 5/296; G16H 50/20

(52) U.S. Cl. CPC A61B 5/389; A61B 5/256; A61B 5/296; A61B 5/6804; A61B 5/725; G16H 50/20; A61B 2560/0468

(57) ABSTRACT

An electromyography (EMG) measurement device includes a garment configured to be worn on an anatomical region of an associated wearer, a plurality of electrodes arranged on the garment to contact skin of the anatomical region when the garment is worn on the anatomical region of the associated wearer, electronics operatively connected with the plurality of electrodes and configured to measure EMG data emanating from the anatomical region, and an electronic processor programmed to derive a contribution of spatial muscle activity of a target muscle or muscle group to the measured EMG data.

This application claims the benefit of U.S. provisional application Ser. No. 63/650,471 filed May 22, 2024, which is incorporated herein by reference in its entirety.

### BACKGROUND

The following relates to the electromyography arts, neuromuscular electrical stimulation arts, neuromuscular therapy arts, neuromuscular rehabilitation arts, virtual reality arts, augmented reality arts, and to the like.

The following relates to improvements in electromyography (EMG) measurement and analysis, and to applications of same in diverse fields such as: neuromuscular electrical stimulation (NMES) guided by EMG signals; EMG-based assessment of neuromuscular debilitation due to spinal cord injury (SCI), stroke, traumatic brain injury (TBI), or other pathologies such as Parkinson's disease; neuromuscular therapy and/or rehabilitation performed using or guided by EMG measurements; EMG-guided muscle tremors suppression; virtual reality (VR) or augmented reality (AR) systems utilizing EMG measurements to monitor participant activity and/or guide VR or AR content presentation; and like applications.

### BRIEF SUMMARY

In accordance with some illustrative embodiments disclosed herein, an electromyography (EMG) measurement device includes a garment configured to be worn on an anatomical region of an associated wearer, a plurality of electrodes arranged on the garment to contact skin of the anatomical region when the garment is worn on the anatomical region of the associated wearer, electronics operatively connected with the plurality of electrodes and configured to measure EMG data emanating from the anatomical region, and an electronic processor programmed to derive a contribution of spatial muscle activity of a target muscle or muscle group to the measured EMG data.

In accordance with some illustrative embodiments disclosed herein, an EMG measurement method includes measuring EMG data emanating from an anatomical region, and deriving a contribution of spatial muscle activity of a target muscle or muscle group to the measured EMG data.

In accordance with some illustrative embodiments disclosed herein, an electronic processor is programmed to measure EMG data emanating from an anatomical region using electrodes arranged on a garment worn on the anatomical region, and derive a contribution of spatial muscle activity of a target muscle or muscle group to the measured EMG data.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically shows an electromyography (EMG) measurement system, together with an optional neuromuscular electrical stimulation (NMES) capability.

FIG. 2 diagrammatically shows a flowchart of a method for performing the spatial muscle activity derivation of FIG. 1 using average movement mapping.

FIG. 3 diagrammatically shows a flowchart of a method for performing the spatial muscle activity derivation of FIG. 1 by mapping to a system of movement equations.

FIG. 4 diagrammatically shows a flowchart of a method for performing the spatial muscle activity derivation of FIG. 1 by blind source separation mapping.

### DETAILED DESCRIPTION

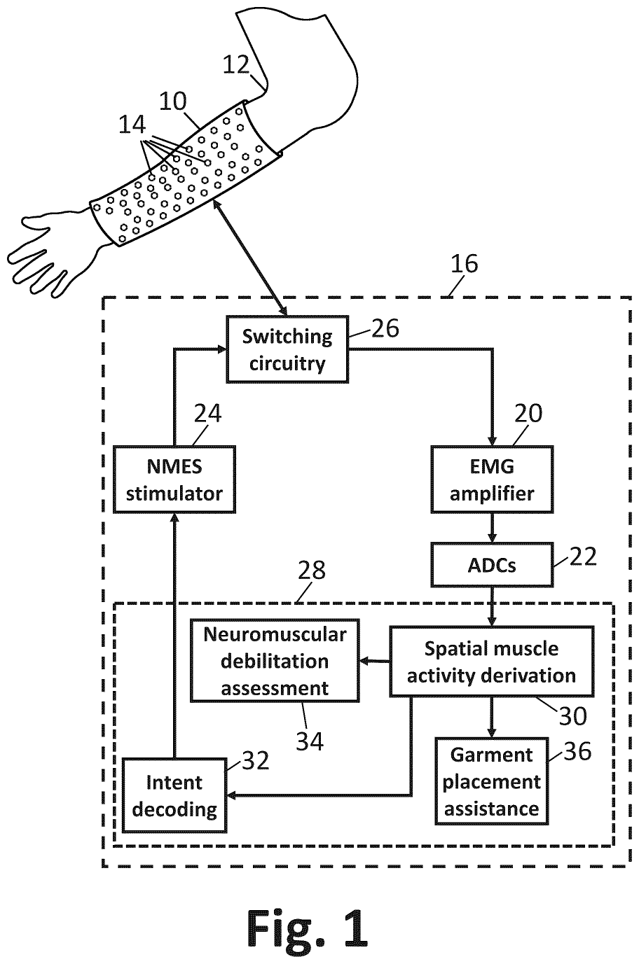

With reference to FIG. 1, an electromyography (EMG) measurement system is shown, which in the illustrative example also includes an optional neuromuscular electrical stimulation (NMES) capability. The EMG measurement system includes a garment 10 that is wearable on an anatomical region 12, and that includes a plurality of electrodes 14 arranged to contact skin of the anatomical region 12 when the garment is worn on the anatomical region. The illustrative garment 10 is a sleeve 10 worn on an arm 12. The garment 10 may be made of a cloth, textile, leather, polyester, or other material, and is sized and shaped to be worn on the anatomical region 12 from which EMG is to be measured. The garment 10 may more generally, for example, be a sleeve that is sized and shaped to be worn on an arm, a leg, a wrist, an ankle, an arm and a wrist, a leg and an ankle, a torso, or so forth. By way of some further examples, suitable garments for a hand would include, for example, a glove or mitten. Suitable garments for a foot would include, for example, a sock or boot. The glove, mitten, sock, or boot can be extended over the wrist or ankle to provide a garment for a wrist and hand or for an ankle and foot, or further extended to provide a garment for an arm and wrist and hand or for a leg and ankle and foot. These are merely non-limiting illustrative examples.

The sizing of the garment 10 is suitably subject-specific to account for different anatomies of different persons; or, the garment 10 may be designed to be adjustable for anatomical differences between persons-for example, the illustrative sleeve 10 could employ a wrap-around arrangement with Velcro that can be adjustably wrapped around arms of different diameters. The plurality of electrodes 14 are disposed on the inside of the garment 10 to contact the skin of the anatomical region 12 when the garment 10 is worn on the anatomical region 12. Note that FIG. 1 illustrates the garment 10 as transparent to reveal the underlying electrodes 14, but more typically the garment will be translucent or opaque. The electrodes 14 are connected by wires (possibly woven into the garment 10), circuitry of flexible printed circuit boards, and/or so forth to connect with associated electronics 16. The various components of the electronics 16 may be integrated with the garment 10, or separate from the garment 10 and connected with the electrodes 14 by suitable electrical wires or cables or the like. Typically, the electrodes 14 are surface electrodes, i.e., transcutaneous electrodes; however, embodying the electrodes 14 as needle electrodes or the like is also contemplated. In some embodiments, the garment 10 is an elastic garment whose elasticity provides compressive force holding the electrodes 14 firmly against the skin of the wearer. Such garment elasticity can also in some specific implementations facilitate the garment 10 being wearable on arms (or other target anatomical region 12) of different sizes. The electrodes 14 are designed to provide good electrical contact with the skin of the anatomical region 12. For example, the electrodes 14 may be electrogel discs, or may comprise an electrically conductive polymer electrode material such as a mixed ionic-electronic conducting (MIEC) material, or so forth. Optionally, the garment 10 may further include other devices such as one or more inertial measurement unit (IMU) devices (not shown) such as an accelerometer, gyroscope, or the like, to provide information on the spatial orientation of the sleeve 10 (and hence of the anatomical region 12).

The electrodes 14 are used to measure EMG signals produced by the anatomical region 12. The EMG signal measurements are potential difference measurements between pairs of electrodes 14, where the pairs of electrodes are pairs of electrodes of the array or, in a monopolar configuration, each pair is an electrode of the array and a common reference electrode. Each such pair of electrodes is referred to herein as an EMG channel. To this end, the electronics 16 include an EMG amplifier 20, which may for example comprise an operational amplifier (op-amp) based amplifier circuit. It will be appreciated that the EMG amplifier 20 is a multi-channel amplifier, e.g. each EMG channel (corresponding to an electrode 14: reference-electrode pair, or to a pair of electrodes 14) is separately received and amplified in parallel by the multi-channel EMG amplifier 20. Preferably, the outputs of the multichannel EMG amplifier 20 are digitized by analog-to-digital converters (ADCs) 22. By way of nonlimiting illustrative example, the combination of the multichannel EMG amplifier 20 and multichannel ADC 22 can be embodied as an Intan EMG amplifier (available from Intan Technologies, Los Angeles, California, USA).

The measured EMG can be utilized in various ways. For example, if the subject is suffering neuromuscular debilitation due to spinal cord injury (SCI), stroke, traumatic brain injury (TBI), or pathologies such as Parkinson's disease, then the EMG can be used to assess the extent to which the motor cortex of the subject's brain is able to transmit motor control neural signals to muscles of the anatomical region 12, and the accuracy of such neural signal transmission if present (e.g., if the subject's volition is to move the index finger then do the transmitted neural signals reach the muscles that cause movement the of the index finger, or are the neural signals mis-transmitted to different muscles due to the neuromuscular debilitation). As another example, if the EMG measurement system is deployed in a virtual reality (VR) or augmented reality (AR) system, then the measured EMG can be used to monitor participant activity, guide the VR or AR content presentation, or so forth. These are some nonlimiting illustrative examples of uses the measured EMG in various applications.

With continuing reference to FIG. 1, the EMG measurement system optionally further includes NMES capability, that is, the ability to apply neuromuscular electrical stimulation to the anatomical region 12 using the electrodes 14. To this end, the electronics 16 further include an NMES stimulator 24. NMES may be applied for various reasons, such as (but not limited to): providing functional electrical stimulation (FES); suppressing muscular tremors; promoting regeneration of damaged nerves; inducing somatosensation (e.g., the sensation of touch, raindrops, an arachnid crawling across the skin, or so forth); and/or et cetera. The configuration of the applied NMES (e.g., which subset of the electrodes 14 apply the NMES, the magnitude of the applied NMES, which may vary spatially over the skin of the anatomical region 12, and so forth) may optionally be guided by the measured EMG, after suitable analysis of the EMG. For example, an SCI patient may have residual motor neuron connectivity between the motor cortex and the musculature of the target anatomy 12, but this motor neuron connectivity may be insufficient to cause the muscle contraction necessary for volitional control of the anatomical region 12. In such a case, the residual motor neuron connectivity may be detected as measured EMG at the muscles intended to be contracted, and FES can then be applied to cause the muscles to actually contract thereby moving the anatomical region 12 in accordance with the volitional intent of the wearer of the garment 10.

To implement the optional NMES capability, the electronics 16 further includes an NMES stimulator 24. To enable switching between applying NMES using the NMES stimulator 24 and receiving EMG measurements via the EMG amplifier 20, suitable switching circuitry 26 is provided, including solid state relays, high voltage field effect transistor (FET) components, and so forth, to enable the same set of electrodes 14 to switch between applying NMES stimulation and measuring EMG. (It is noted that if the EMG measurement system does not include NMES capability, then both the NMES stimulator 24 and the switching circuitry 26 may be omitted.) to perform NMES, the NMES stimulator 24 generates suitable electrical pulses that are applied to the anatomical region 12 (or a selected portion thereof) by a selected subset of the electrodes 14. In some nonlimiting illustrative embodiments, the applied NMES may comprise NMES pulse waveforms including monophasic and/or biphasic pulses with a voltage between 80 to 300 Volts inclusive or higher. In one specific example, the NMES pulse waveform is a monophasic pulse with a peak current of 0-20 mA which is modulated to vary strength of muscle contraction, frequency of 50 Hz, and a pulse width duration of 500 ms. Again, these are non-limiting illustrative examples. Analogously to the EMG amplifier 20, it will be appreciated that the NMES stimulator 24 is a multichannel NMES stimulator that can in general independently apply different NMES to different channels (where a channel corresponds to an electrode 14: reference-electrode pair, or to a pair of electrodes 14).

With continuing reference to FIG. 1, the electronics 16 further include a computer, microprocessor, or other electronic processor 28 that is programmed to perform spatial muscle activity derivation 30 to extract information from the EMG data about the muscle activity that produced the EMG data, e.g. by deriving the contribution of individual muscles or muscle groups to the EMG data.

The derived muscle activity can be used in various ways. In the illustrative example of FIG. 1, two nonlimiting examples of applications utilizing the derived muscle activity are shown. In one example, the electronic processor 28 is further programmed to perform intent decoding processing 32 to determine volitional intent of the user (i.e., the person wearing the garment 10). In this example application, the user may be an SCI patient who has residual motor neuron connectivity between the user's motor cortex and the user's musculature of the target anatomy 12, but this residual motor neuron connectivity is insufficient to cause the muscle contraction necessary for volitional control of the anatomical region 12. The intent decoding 32 determines the user's volitional intent by detecting which muscles or muscle groups are receiving the residual motor neuron signals (as manifested by the muscle activity derived from the EMG data by the processing 30), and the NMES stimulator 24 then applies functional electrical stimulation to the identified muscles via the electrodes 14 to cause the anatomical region 12 to perform the movement volitionally intended by the user.

In another illustrative application utilizing the derived muscle activity, the electronic processor 28 is further programmed to perform neuromuscular debilitation assessment 34. This can take various forms. For example, in one approach the user (who is suffering from neuromuscular debilitation due to SCI, stroke, TBI, or another pathology such as Parkinson's disease) is asked to perform a movement of the anatomical region 12. The user makes the effort but is unable to perform the movement, or performs the movement poorly. The muscle activity derived from the EMG data by the processing 30 during this effort is processed by the assessment processing 34 to determine the strength of motor neural signals delivered to the anatomical region 12 during the user's effort, as well as information on how accurately those motor neural signals are targeted to the correct (versus incorrect) muscles. As some further nonlimiting illustrative applications, the derived muscle activity can also provide physiological interpretation to predicted neuromuscular assessment output. For example, if it says quantitatively, a stroke subject has limited mobility, then the derived muscle activity could highlight deficient muscles.

In yet another illustrative application utilizing the derived muscle activity, the electronic processor 28 is further programmed to perform garment placement assistance 36 to determine (and optionally correct for) a placement of the garment 10 on the anatomical region 12. For example, to use the garment 10 for NMES, it must be determined where to stimulate muscles, and this varies between individuals, sleeve and electrode positioning, and individual neuro-cognitive impairment, as well as any variations in placement of the garment 10 on the anatomical region 12 (which can vary from sessions to session even for a single individual, depending on the fitting of the garment). To use the array of electrodes 14 as an assistive or rehabilitation device employing NMES, initial calibration data is acquired and used to train machine learning algorithms of the intent decoding 32 to decode a user's intention. However, accuracy of the decoder 32 may be degraded by variability in positioning of the garment 10 on the anatomy 12, and by individual muscle activation patterns requiring additional calibration data.

By automatically determining the placement of the garment 10 on the anatomy 12 for the current session through the muscle mapping 30, a suitable spatial garment (mis) placement correction can be made so that the data from previous sessions may be used quickly to calibrate the system for a current session. For example, the previous calibration may be used as a starting point for calibrating the NMES for the current session, shifted to correct for any misplacement of the garment 10 on the anatomy 12 determined by the garment placement assistance 36.

To illustrate, consider an example formulation in which the position of each of the electrodes 14 is specified in cylindrical coordinates (r, θ) where r is longitudinal position along the arm (in the direction running from the wrist to the elbow or vice versa) and θ is a circumferential position around the arm (measured from a reference designated as θ=0). In the previous calibration, a target muscle (or muscle group) to be targeted by NMES was determined to have a location (r_{mus,0}, θ_{mus,0}). If in a new session the spatial muscle activity derivation 30 determines the target muscle (or muscle group) is now at a shifted location (r_{mus,0}+Δr, θ_{mus,0}+Δθ), this is likely due to a different placement (i.e., a misplacement) of the garment 10 for the current session compared with the previous calibration. To compensate for this (mis) placement, the previous calibration can be spatially shifted by the determined shift (Δr, Δθ) to provide a more accurate starting point for update calibrating (i.e., tuning of the calibration) for the current session. By performing such a shift mathematically there is no need for the user to reposition the garment 10 on the anatomy 12, and the previous calibration can be used as the starting point for calibration update (i.e., tuning) without such repositioning.

The applications 32, 34, and 36 diagrammatically shown in FIG. 1 are nonlimiting examples. More generally, the muscle activity derived from the EMG data by the processing 30 can be used in various applications such as: EMG-guided NMES (i.e., using volitional intent obtained from the measured EMG via intent decoding 32); EMG-based assessment 34 of neuromuscular debilitation due to SCI, stroke, TBI, Parkinson's disease, or so forth; neuromuscular therapy and/or rehabilitation performed using or guided by EMG measurements; EMG-guided muscle tremors suppression; VR or AR systems utilizing EMG measurements to monitor participant activity and/or guide VR or AR content presentation; and like applications.

The processing 30 to derive muscle activity from the EMG data is challenging. Large arrays of electrodes 14 spanning the skin over many muscles can, in principle, be used to calculate muscle synergies and the spatiotemporal activation patterns of multiple muscles. However, identifying individual muscle contribution from a large forearm array (e.g., as shown for the illustrative sleeve 10 of FIG. 1) can be impacted by a variety of individual morphological differences such as forearm size, and entails separating overlapping and nearby muscles. Furthermore, electrical activity from muscles propagates across the skin due to conductivity of the skin (and, in some embodiments, with further contribution due to an optional conductive medium applied to the anatomical region 12 and/or to an inner surface of the garment 10 to reduce electrode-to-skin resistance). Due to propagation of the EMG signals across the skin, EMG measured by the EMG channels is subject to cross-talk, such that multiple channels may record EMG from the same muscle. Disentangling the contribution of individual muscles or muscle groups to the recorded EMG signal is thus challenging.

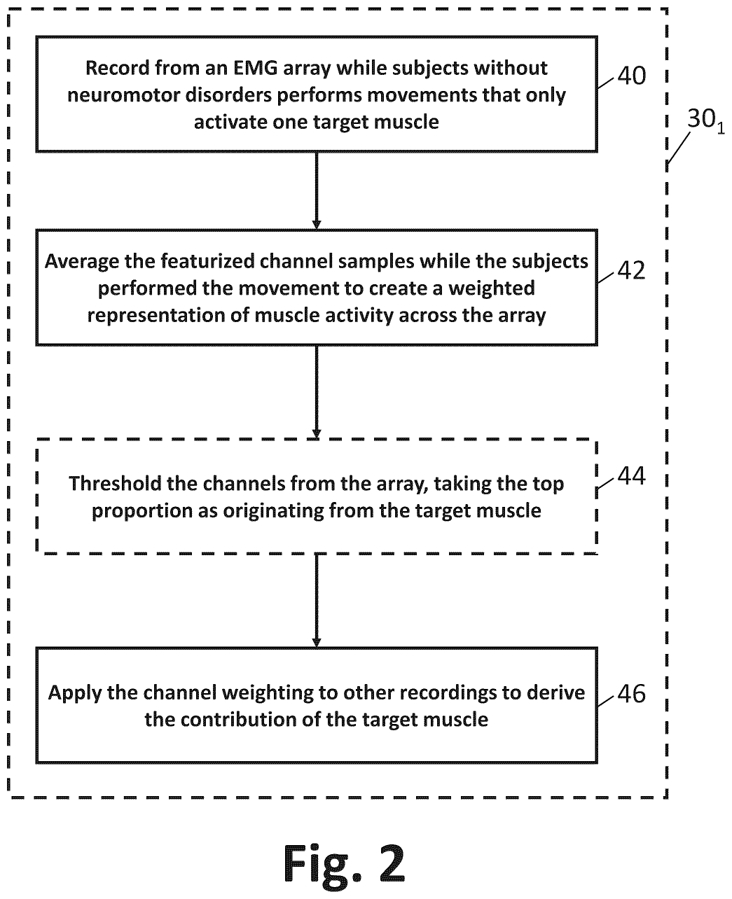

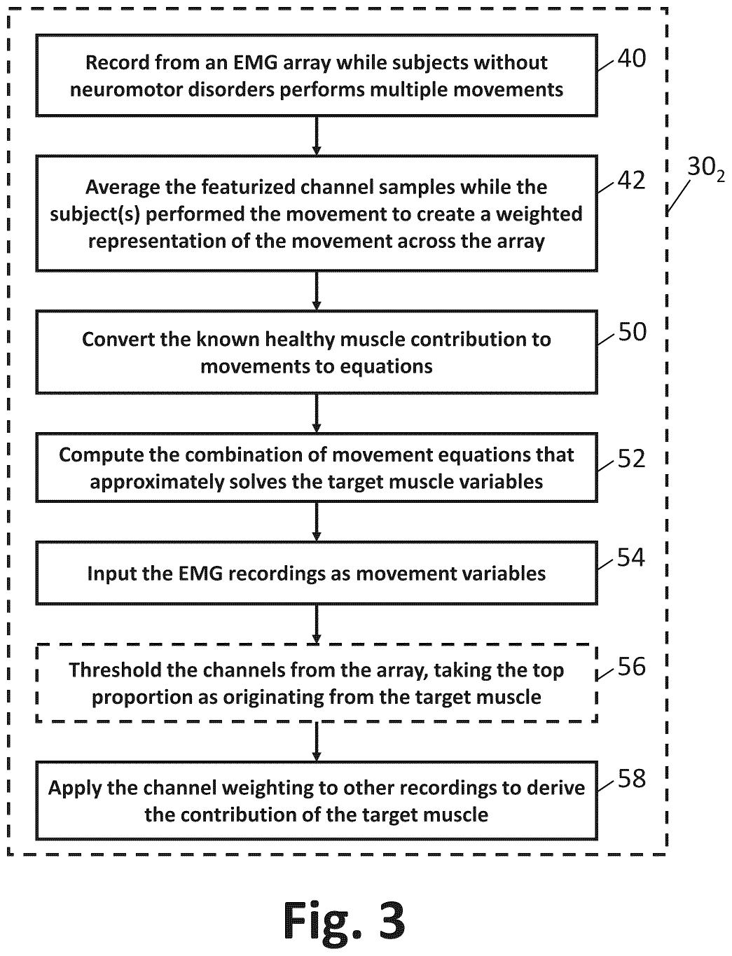

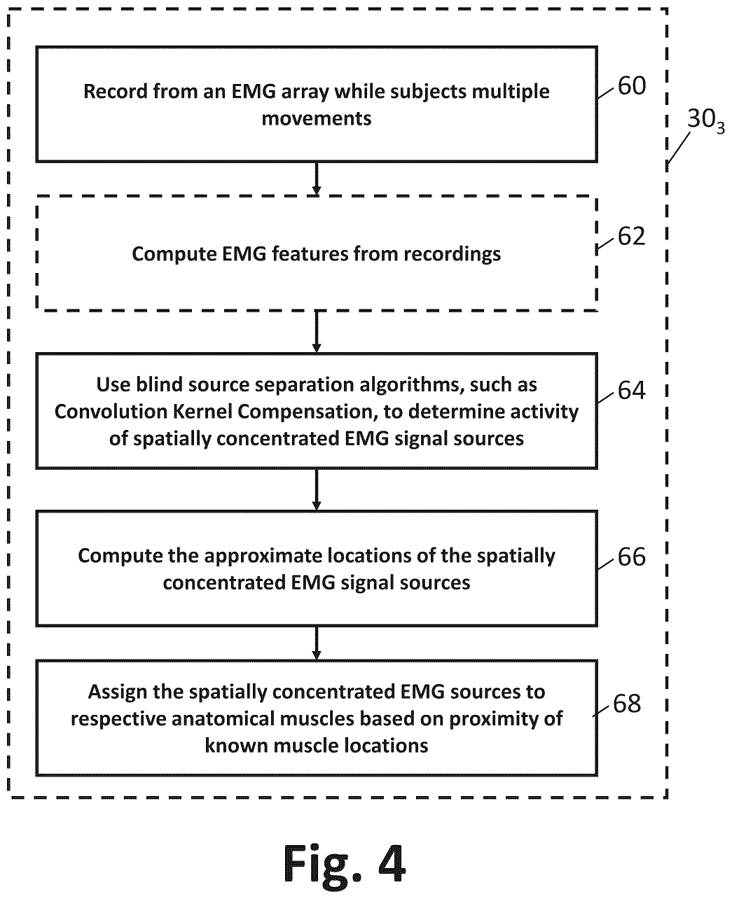

Using various approaches disclosed herein, muscle mapping and contribution can be derived from EMG data measured using the array of electrodes 14. The various approaches disclosed herein include average movement mapping approaches (e.g., see FIG. 2), mapping to a system of movement equations (e.g., see FIG. 3), or blind source separation (BSS) mapping (e.g., see FIG. 4). These approaches can be used to retroactively determine muscle contributions assuming only similar muscle anatomy and consistent placement of the array of electrodes 14 on the anatomical region 12.

The disclosed approaches for muscle mapping use EMG data alone to determine the spatial map of individual muscles or muscle groups. The approaches employing average movement mapping (FIG. 2) or mapping to a system of movement equations (FIG. 3) include a training phase in which training EMG data are measured for musculature of persons with a healthy anatomical region 12 (so that it is known a priori which muscles are active during the movement) and a muscle map is created by combining this training data to maximize the expected contribution muscles of interest. In a subsequent inference phase, the created muscle map is applied to a new session and/or new subject (healthy or with neuromuscular disorder) to map the EMG to muscles or muscle groups.

Approaches employing BSS mapping (FIG. 4) do not utilize a prior training dataset. Rather, BSS is used to identify and locate sources of EMG signals, effectively finding the map of muscles.

With reference now to FIG. 2, a mapping embodiment 30₁ of muscle mapping 30 is described, which employs average movement mapping. In an operation 40, EMG is recorded (i.e., measured) using the array of electrodes 14 while subjects without neuromotor disorders perform movements that only activate one target muscle or muscle group. To provide a large and diverse training dataset, the operation 40 may acquire training EMG data from a plurality of different healthy training subjects (i.e., subjects without neuromotor disorders), and may optionally do so over a plurality of sessions for each training subject. The operation 40 may also include processing of the EMG data to derive features with the output referred to here as featurized EMG channel samples. By way of nonlimiting illustrative example, the derived features could include one or more of: root mean square (RMS), mean absolute value (MAV), wavelength (WL), tangent space (TS), and so forth. In an operation 42, the featurized EMG channel samples while the subjects performed the movement using the target muscle or muscle group are averaged to create a weighted representation of muscle activity of the target muscle or muscle group across the array of electrodes 14. The weighted representation of muscle activity of the target muscle could be, in one nonlimiting illustrative example, a two-dimensional (2D) Gaussian distribution of EMG activity across the electrodes spanning the spatial area of the muscle or muscle group, with the weights being the variance, standard deviation, or the like. In another nonlimiting illustrative example, the weighted representation of muscle activity of the target muscle could be constructed as the average across all subjects. This would then create regions of high/low weight based on how many subjects activated the same spatial area. Through this overlap, minor sleeve positioning discrepancies cancel out. In an optional operation 44, the featurized EMG channels may be thresholded, taking the top proportion as originating from the target muscle or muscle group. This completes the training phase 40, 42, 44.

Thereafter, in a current session, the channel weighting output by the training phase 40, 42, 44 is applied in an operation 46 to recordings of EMG data acquired of a current subject in a current session to derive the contribution of the target muscle or muscle group to the EMG data. Advantageously, since the training data acquired and averaged in the training operations 40 and 42 was for a plurality of individuals and/or multiple sessions, this training EMG data (and the resulting weighted representation of the muscle activity of the target muscle or muscle group) has a statistical spread that accommodates variability of the location of the target muscle or muscle group amongst individuals; and that accommodates variability in the placement of the garment 10 on the anatomical region 12 amongst sessions.

In summary, the mapping embodiment 301 derives the contribution of spatial muscle activity of the target muscle or muscle group to the measured EMG data acquired in the current session by operations including: averaging (operation 42) training EMG data acquired (operation 40) from a plurality of training subjects while performing a movement that only activates the target muscle or muscle group to generate a weighted representation of muscle activity of the target muscle or muscle group; and deriving (operation 46) the contribution of the spatial muscle activity of the target muscle or muscle group to the measured EMG data by applying weightings of the weighted representation of muscle activity of the target muscle or muscle group to the measured EMG data.

While the foregoing operations 40, 42, 44, 46 are described for a single target muscle or muscle group, it will be appreciated that the training operations 40, 42, 44 can be repeated for a plurality of different target muscles or muscle groups to provide a weighted EMG representation for each target muscle or muscle group. Then, in the operation 46 these weighted representations can be used to map the EMG data acquired in the current session to the various target muscles or muscle groups.

With reference now to FIG. 3, a mapping embodiment 302 of muscle mapping 30 is described, which performs the mapping to a system of movement equations. The operation 40 already described with reference to FIG. 2 is performed in this approach as well, in order to collect training EMG data which may be from a plurality of subjects and/or over a plurality of sessions. The mapping embodiment 302 of FIG. 3 also performs the operation 42 of FIG. 2, in which the featurized EMG channel samples acquired while the subjects performed the movement using the target muscle or muscle group are averaged to create a weighted representation of muscle activity of the target muscle or muscle group across the array of electrodes 14. In an operation 50, the known healthy contribution to the movement using the target muscle or muscle group is converted to one or more equations.

A nonlimiting illustrative example of the operation 50 is as follows. A movement equation for wrist extension (WE) can be written as:

$$WE = Extensor Carpi Ulnaris + Extensor Carpi Radialis ( Brevis + Longus ) + Extensor Digitorum Minimi \qquad ( 1 )$$

A movement equation for wrist flexion (WF) can be written as:

$$WF = F lexor Carpi Ulnaris + Flexor Carpi Radialis + Palmaris Longus \qquad ( 2 )$$

A movement equation for ulnar deviation (UD) can be written as:

$$UD = Flexor Carpi Ulnaris + Extensor Carpi Ulnaris \qquad ( 3 )$$

A movement equation for radial deviation (RD) can be written as:

$$RD = Flexor Carpi Radialis + Extensor Carpi Radialis ( Brevis + Longus ) \qquad ( 4 )$$

A muscle equation derivation example is then given as the set of equations:

$$\begin{matrix}Eq . 1 & \\ WE + UD = 2 ECU + ECR + EDM + FCU & ( 5 )\end{matrix}$$

$$WF + RD = ECR + FCU + 2 FCR + PL \qquad Eq . 2$$

$$Eq . 1 - Eq . 2 = 2 ECU + EDM - 2 FCR - PL \qquad Eq . 3$$

where ECU˜=Threshold[Eq. 1, Eps], Eps=max (Eq. 3)/2. This is merely one nonlimiting illustrative example, and more generally the operation 50 converts the known healthy contribution to the movement using the target muscle or muscle group to one or more equations based on the movements and underlying musculoskeletal anatomy.

In an operation 52, the combination of movement equations is computed that approximately solves the target muscle variables. In one nonlimiting illustrative example, each muscle group that is intended to match to the full EMG signal is set as an unknown variable. Then with a system of equations based on known activity of certain regions, the unknown target muscle variables are computed (e.g. through combinatorial movements). This completes the training phase 40, 42, 50, 52.

Thereafter, in an operation 54 of a current session, EMG recordings are input as movement variables. In an optional operation 56, the EMG channels are thresholded, taking the top proportion as originating from the target muscle. In an operation 58, the EMG channel weighting is applied to other recordings to derive the contribution of the target muscle. Advantageously, since the training data acquired and averaged in the training operations 40 and 42 was for a plurality of individuals and/or multiple sessions, this training EMG data (and the resulting movement equation representing the muscle activity of the target muscle or muscle group) has a statistical spread that accommodates variability of the location of the target muscle or muscle group amongst individuals; and that accommodates variability in the placement of the garment 10 on the anatomical region 12 amongst sessions.

In summary, the mapping embodiment 302 derives the contribution of spatial muscle activity of the target muscle or muscle group to the measured EMG data acquired in the current session by operations including: averaging (operation 42) training EMG data acquired (operation 40) from a plurality of training subjects while performing a movement that only activates the target muscle or muscle group to generate a weighted representation of muscle activity of the target muscle or muscle group; converting (operations 50 and 52) the weighted representation of muscle activity of the target muscle or muscle group to at least one movement equation representing the muscle activity of the target muscle or muscle group; and deriving (operations 56 and 58) the contribution of the spatial muscle activity of the target muscle or muscle group to the measured EMG data (from operation 54) by applying the at least one movement equation representing the muscle activity of the target muscle or muscle group to the measured EMG data.

While the foregoing operations 40, 42, 50, 52, 54, 56, and 58 are described for a single target muscle or muscle group, it will be appreciated that the training operations 40, 42, 50, 52 can be repeated for a plurality of different target muscles or muscle groups to provide an equation representation for each target muscle or muscle group. Then, in the operations 54, 56, and 58 these equation representations can be used to map the EMG data acquired in the current session to the various target muscles or muscle groups.

With reference now to FIG. 4, a mapping embodiment 303 of muscle mapping 30 is described, which uses blind source separation (BSS) mapping. Unlike the embodiments 301 and 302, no training phase is involved with this approach. Rather, in an operation 60 EMG data is recorded from a subject in a current session. This EMG data may represent multiple movements involving multiple target muscles or muscle groups. In an optional operation 62, EMG features may be computed from the recorded EMG data. In an operation 64, a BSS algorithm such as Convolutional Kernel Compensation is used to determine activity of spatially concentrated EMG signal sources (where each spatially concentrated EMG signal source is expected to correspond to a specific muscle or muscle group). In an operation 66, the approximate locations of the spatially concentrated EMG signal sources are computed. In one approach, a BSS algorithm is used to determine activity of spatially concentrated EMG signal sources. To get spatial information, the inverse (i.e., backward) filters computed via the BSS algorithm are used to reconstruct the original signal from source. This provides the physiological interpretation of the spatial distribution of each source. In the example of a forearm sleeve garment 10 of FIG. 1, for example, the approximate locations may be in the cylindrical coordinates system (r, θ) previously described. In an operation 68, the spatially concentrated EMG signal sources are assigned to respective anatomical muscles or muscle groups based on proximity of known muscle (or muscle group) locations (e.g., known from an anatomical atlas or the like).

In summary, the mapping embodiment 303 derives the contribution of spatial muscle activity of the target muscle or muscle group to the measured EMG data acquired in the current session by operations including: determining activity of spatially concentrated EMG signal sources in the measured EMG data using a blind source separation (BSS) algorithm to the measured EMG data (operation 64); computing locations of the spatially concentrated EMG signal sources determined using inverse (i.e., backward) filters computed via the BSS algorithm (operation 66); and deriving the contribution of the spatial muscle activity of the target muscle or muscle group to the measured EMG data based on the spatially concentrated EMG signal source in closest proximity to the target muscle or muscle group (operation 68).

In the illustrative example, the operation 64 employs Convolution Kernel Compensation as the BSS algorithm. More generally, however, the operation 64 may employ any suitable BSS algorithm, such as another Convolutive BSS algorithm, or Independent Component Analysis (ICA), or a BSS algorithm using approximate joint diagonalization of covariance (AJDC) matrices.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. An electromyography (EMG) measurement device comprising:
a garment configured to be worn on an anatomical region of an associated wearer;
a plurality of electrodes arranged on the garment to contact skin of the anatomical region when the garment is worn on the anatomical region of the associated wearer;
electronics operatively connected with the plurality of electrodes and configured to measure EMG data emanating from the anatomical region; and
an electronic processor programmed to derive a contribution of spatial muscle activity of a target muscle or muscle group to the measured EMG data.

2. The EMG measurement device of claim 1, wherein the electronic processor is programmed to derive the contribution of the spatial muscle activity of the target muscle or muscle group to the measured EMG data by operations including:
averaging training EMG data acquired from a plurality of training subjects while performing a movement that only activates the target muscle or muscle group to generate a weighted representation of muscle activity of the target muscle or muscle group; and
deriving the contribution of the spatial muscle activity of the target muscle or muscle group to the measured EMG data by applying weightings of the weighted representation of muscle activity of the target muscle or muscle group to the measured EMG data.

3. The EMG measurement device of claim 1, wherein the electronic processor is programmed to derive the contribution of the spatial muscle activity of the target muscle or muscle group to the measured EMG data by operations including:
averaging training EMG data acquired from a plurality of training subjects while performing a movement that only activates the target muscle or muscle group to generate a weighted representation of muscle activity of the target muscle or muscle group;
converting the weighted representation of muscle activity of the target muscle or muscle group to at least one movement equation representing the muscle activity of the target muscle or muscle group; and
deriving the contribution of the spatial muscle activity of the target muscle or muscle group to the measured EMG data by applying the at least one movement equation representing the muscle activity of the target muscle or muscle group to the measured EMG data.

4. The EMG measurement device of claim 1, wherein the electronic processor is programmed to derive the contribution of the spatial muscle activity of the target muscle or muscle group to the measured EMG data by operations including:
determining activity of spatially concentrated EMG signal sources in the measured EMG data using a blind source separation (BSS) algorithm to the measured EMG data;
computing locations of the spatially concentrated EMG signal sources determined using inverse filters computed via the BSS algorithm; and
deriving the contribution of the spatial muscle activity of the target muscle or muscle group to the measured EMG data based on the spatially concentrated EMG signal source in closest proximity to the target muscle or muscle group.

5. The EMG measurement device of claim 4, wherein the BSS algorithm is a Convolutive BSS algorithm.

6. The EMG measurement device of claim 1, further comprising:
a neuromuscular electrical stimulation (NMES) stimulator operatively connected with the electrodes;
wherein the electronic processor is further programmed to operate the NMES stimulator to deliver NMES to the target muscle or muscle group based on the derived contribution of the spatial muscle activity of the target muscle or muscle group to the measured EMG data.

7. The EMG measurement device of claim 1, wherein the electronic processor is programmed to derive contributions of spatial muscle activity of a plurality of target muscles or muscle groups to the measured EMG data, and is further programmed to:
perform a neuromuscular debilitation assessment based on the derived contributions of spatial muscle activity of the plurality of target muscles or muscle groups to the measured EMG data.

8. The EMG measurement device of claim 1, wherein the electronic processor is further programmed to:
determine a placement shift of the garment on the anatomical region compared with a previous calibration based on a shift of a location of the contribution of spatial muscle activity of a target muscle or muscle group to the measured EMG data compared with a previous location of the target muscle or muscle group during the previous calibration.

9. An electromyography (EMG) measurement method comprising:
measuring EMG data emanating from an anatomical region; and
deriving a contribution of spatial muscle activity of a target muscle or muscle group to the measured EMG data.

10. The EMG measurement method of claim 9, wherein the deriving includes:
averaging training EMG data acquired from a plurality of training subjects while performing a movement that only activates the target muscle or muscle group to generate a weighted representation of muscle activity of the target muscle or muscle group; and
deriving the contribution of the spatial muscle activity of the target muscle or muscle group to the measured EMG data by applying weightings of the weighted representation of muscle activity of the target muscle or muscle group to the measured EMG data.

11. The EMG measurement method of claim 9, wherein the deriving includes:
averaging training EMG data acquired from a plurality of training subjects while performing a movement that only activates the target muscle or muscle group to generate a weighted representation of muscle activity of the target muscle or muscle group;
converting the weighted representation of muscle activity of the target muscle or muscle group to at least one movement equation representing the muscle activity of the target muscle or muscle group; and
deriving the contribution of the spatial muscle activity of the target muscle or muscle group to the measured EMG data by applying the at least one movement equation representing the muscle activity of the target muscle or muscle group to the measured EMG data.

12. The EMG measurement method of claim 9, wherein the deriving includes:
determining activity of spatially concentrated EMG signal sources in the measured EMG data using a blind source separation (BSS) algorithm to the measured EMG data;
computing locations of the spatially concentrated EMG signal sources determined using inverse filters computed via the BSS algorithm; and
deriving the contribution of the spatial muscle activity of the target muscle or muscle group to the measured EMG data based on the spatially concentrated EMG signal source in closest proximity to the target muscle or muscle group.

13. The EMG measurement method of claim 12, wherein the BSS algorithm is a Convolutive BSS algorithm.

14. The EMG measurement method of claim 9, further comprising:
performing spatial muscle mapping to determine a sleeve shift and automatically updating NMES stimulation patterns based on the sleeve shift; and
delivering neuromuscular electrical stimulation (NMES) to the target muscle or muscle group based on the derived contribution of the spatial muscle activity of the target muscle or muscle group to the measured EMG data.

15. The EMG measurement method of claim 9, wherein the deriving includes deriving contributions of spatial muscle activity of a plurality of target muscles or muscle groups to the measured EMG data, and the method further comprises:
performing a neuromuscular debilitation assessment based on the derived contributions of spatial muscle activity of the plurality of target muscles or muscle groups to the measured EMG data.

16. The EMG measurement device of claim 9, wherein the EMG data emanating from the anatomical region is measured using electrodes arranged on a garment worn on the anatomical region, and the method further comprises:
determine a placement shift of the garment on the anatomical region compared with a previous calibration based on a shift of a location of the contribution of spatial muscle activity of a target muscle or muscle group to the measured EMG data compared with a previous location of the target muscle or muscle group during the previous calibration.

17. An electronic processor programmed to:
measure EMG data emanating from an anatomical region using electrodes arranged on a garment worn on the anatomical region; and
derive a contribution of spatial muscle activity of a target muscle or muscle group to the measured EMG data.

18. The electronic processor of claim 17, wherein the deriving includes:
averaging training EMG data acquired from a plurality of training subjects while performing a movement that only activates the target muscle or muscle group to generate a weighted representation of muscle activity of the target muscle or muscle group; and
deriving the contribution of the spatial muscle activity of the target muscle or muscle group to the measured EMG data by applying weightings of the weighted representation of muscle activity of the target muscle or muscle group to the measured EMG data.

19. The electronic processor of claim 17, wherein the deriving includes:
averaging training EMG data acquired from a plurality of training subjects while performing a movement that only activates the target muscle or muscle group to generate a weighted representation of muscle activity of the target muscle or muscle group;
converting the weighted representation of muscle activity of the target muscle or muscle group to at least one movement equation representing the muscle activity of the target muscle or muscle group; and
deriving the contribution of the spatial muscle activity of the target muscle or muscle group to the measured EMG data by applying the at least one movement equation representing the muscle activity of the target muscle or muscle group to the measured EMG data.

20. The electronic processor of claim 17, wherein the deriving includes:
determining activity of spatially concentrated EMG signal sources in the measured EMG data using a blind source separation (BSS) algorithm to the measured EMG data;
computing locations of the spatially concentrated EMG signal sources determined using the BSS algorithm; and
deriving the contribution of the spatial muscle activity of the target muscle or muscle group to the measured EMG data based on the spatially concentrated EMG signal source in closest proximity to the target muscle or muscle group.
